(19) United States
(12) Patent Application Publication
(10) Pub. No.: US 2020/0215133 A1
(43) Pub. Date: Jul. 9, 2020

(54) COMBINATION OF IMMUNO-ONCOLYTIC VIRUS DRUGS FOR ENHANCING SYSTEMIC IMMUNE RESPONSE AND APPLICATION THEREOF

(71) Applicant: SICHUAN ONCOCARE BIOPHARMACEUTICAL, INC., Chengdu (CN)

(72) Inventor: Li YU, Chengdu (CN)

(21) Appl. No.: 16/752,328

(22) Filed: Jan. 24, 2020

Related U.S. Application Data
(63) Continuation of application No. PCT/CN2019/098798, filed on Aug. 1, 2019.

(30) Foreign Application Priority Data
Jan. 7, 2019 (CN) 201910012374.8

(51) Int. Cl. A61K 35/768; C12N 7/00; A61K 9/00

(52) U.S. Cl. CPC A61K 35/768; C12N 2770/24132; A61K 9/0019; C12N 7/00

(57) ABSTRACT

This disclosure provides the innovation of a group of flaviviruses carrying respective lymphocyte genes that results in targeting specific T cells of immune system to therapy solid cancers and provides strategies for using these oncolytic viruses to reduce or to avoid immune resistance to single virus treatment and thus increase efficiency against cancer.

### CROSS-REFERENCE TO RELATED APPLICATION

This is a continuation application of International Application No. PCT/CN2019/098798, filed on Aug. 1, 2019, which claims priority to Chinese Patent Application No. 201910012374.8 filed on Jan. 7, 2019, the entire contents of which are incorporated herein by reference.

### TECHNICAL FIELD

The present disclosure relates to the field of biopharmaceutical and cancer immunotherapy.

### BACKGROUND

Cancer Statistics and Immunotherapy

Cancer has a major impact on society across the world and in the United States. According to WHO report, cancer is a leading cause of death worldwide and is responsible for an estimated 9.6 million deaths in 2018. Globally, about 1 in 6 deaths is due to cancer. Approximately 70% of deaths from cancer occur in low- and middle-income countries. Statistics analysis by NIH NCI estimated 1,735,350 new cases of cancer were diagnosed in the United States and 609,640 people died from the disease in 2018. Furthermore, the number of new cancer cases is on the rise globally. The number of new cancer cases per year is expected to rise to 23.6 million by 2030. The number of global cancer deaths is projected to increase by 45% between 2008 and 2030. And deaths from cancer worldwide are projected to reach over 13 million in 2030.

The most common cancers worldwide are: Lung (2.09 million cases); breast (2.09 million cases); colorectal (1.80 million cases); prostate (1.28 million cases); skin cancer (non melanoma) (1.04 million cases); stomach (1.03 million cases).

The most common causes of cancer death are cancers of: Lung (1.76 million deaths); colorectal (862 000 deaths); stomach (783 000 deaths); liver (782 000 deaths); breast (627 000 deaths). It is notable that the high mortality of cancer is due to mostly delaying in early diagnoses and failing at available treatments with modern medical technology. Thus, development of more effective medicine to treat cancer is very necessary to lift up the level of human health and living quality. In the past ten years, variety of immunotherapy for cancer has been developed. Some were licensed, including monoclonal antibodies (e.g. ipilimumab); chimeric antigen receptor-T cell therapy (CAR-T); and oncolytic viruses (e.g. T-Vec).

Oncolytic Viruses

Currently, there were more than 25 products of oncolytic viruses (OVs) in the clinical trials worldwide (Ibrahim Ragab Eissa, et al. 2018. The Current Status and Future Prospects of Oncolytic Viruses in Clinical Trials against Melanoma, Glioma, Pancreatic, and Breast Cancers. Cancers (Basel), 10(10): 356). Oncolytic viral therapy is a new promising strategy against cancer. OVs are defined as genetically engineered or naturally occurring viruses that can replicate in cancer cells, leading to lysis of the tumor cells. OVs infect tumor cells and replicate to produce progeny virus particles. They destroy infected cancer cells as release virus particles and repeat this cycle to lysis remaining tumors cells. OVs are therefore a targeted therapy and replicable biotherapeutic drugs. Beside the primary lysis effect, OVs can also stimulate the immune system. Tumors are an immuno-suppressive environment in which the immune system is silenced to not have immune response against cancer cells. Delivery of OVs into the tumor may wake up the immune system so that it may facilitate a strong response not only against the infected virus but also indirectly against tumor cells. The OV lysis of tumor cells can not only release the OV antigen, but also produce or expose cancer-specific antigens. These antigens are required to elicit APC and T cell responses. Both innate and adaptive immune responses contribute to this process by inducing inflammatory factors such as tumor necrosis factor (TNF), interleukin-1β (IL-1β) and complement to producing immune responses against tumor antigens, to increase T-cell infiltration, and to facilitating immunological memory.

Main Defects in Oncolytic Virus Therapy

It is well known that viruses are pathogens and thus naturally antigens or immunogenicity that are recognized by the immune system. The viral infection leads to an immune response, which mainly produces antiviral antibodies and T-cell responses to inhibit viral amplification. This is the same mechanism as human vaccination to prevent infectious diseases, in which the virus immunogenicity plays critical roles to prevent diseases, beneficial to human health. In contrast to the vaccination, however, when using the virus as therapy drugs, its immunogenicity becomes a barrier to block the efficacy of the virus treatment. Since virus replication is inhibited by rapidly elicited immune responses, the use of oncolytic viruses as cancer therapy and/or gene therapy vectors are mostly limited by the existence of preformed immunity in various populations. Therefore, single oncolytic virus treatment cannot reach to expect efficacy even administrates more than twice according to principles of immunology.

The anti-viral response of immune system comes from two aspects: (A) population vaccination (e.g. application of vaccination world wild) and (B) virus epidemic history or personal infection history in the local area (e.g. outbreak of Zika virus infection extended across all Brazilian regions in 2015 and 2016). In the case of (A), for example, approximately 85% of children develop protective antibody levels when given one dose of measles vaccine at nine months of age, and 90% to 95% respond when vaccinated at 12 months of age (Vesikari T, et al. 2012. Immunogenicity and safety of a two-dose regimen of combined measles, mumps, rubella and varicella live vaccine (ProQuad(®)) in infants from 9 months of age. Vaccine. 30(20):3082-3089). The raised immune response to measles by vaccination protects measles infection in whole life.

In case of (B), for example, prevalence of herpes simplex virus type 1 (HSV-1) was 47.8%, and prevalence of herpes simplex virus type 2 (HSV-2) was 11.9% in the USA during 2015-2016 (Geraldine McQuillan, et al. Prevalence of Herpes Simplex Virus Type 1 and Type 2 in Persons Aged 14-49: United States, 2015-2016. CDC USA. NCHS Data Brief No. 304 February, 2018). Adenoviruses are common viruses that cause a range of illnesses. Between 33 and 60% of the U.S. population have neutralization antibody (NAb) to Ad5 as a result of natural infection. While a high rate of NAb to group C adenoviruses (Ad2 and Ad5 included) was demonstrable in infant sera (Edward Nwanegbo, et al. 2004. Prevalence of Neutralizing Antibodies to Adenoviral Serotypes 5 and 35 in the Adult Populations of The Gambia, South Africa, and the United States. Clin Diagn Lab Immunol. 11(2): 351-357). The anti-viral response may manifest as the induction of proinflammatory cytokines, a humoral antibody response that neutralizes the oncolytic virus, and/or a cellular immune response that targets and destroy cells expressing oncolytic viral antigens. Therefore, the consequence of anti-viral response could represent a big hurdle that prevents the effective application of these OVs as therapeutic drugs. Besides virus susceptibility to host defenses, another limitation to use the oncolytic virus as pharmaceuticals is the tissue specificity of virus infection. That is the viral tropism. Viral affinity for specific tissues/cells is determined by (1) accessibility of virus to tissue, (2) cell susceptibility to virus multiplication. Because of the tropism, certain oncolytic viruses may only infect some type of tumor.

It is obvious that these hurdles may explain the lower rate of cancer therapy by using oncolytic viruses (e.g. T-Vec), which has only 16-26% of the objective response rate (ORR) in monotherapy (Robert M. Conry, et al. 2018. Talimogene laherparepvec: First in class oncolytic virotherapy. Hum Vaccin Immunother. 14(4): 839-846). In order to achieve effective treatment of cancer with oncolytic virus, a key element is to avoid the limitation of preformed immune defense again oncolytic virus. It is the certainty that measles virus, VSV, reovirus, and adenovirus type 5 are not good to be oncolytic virus candidates because the human population has already high immunity to these viruses, as described above. Therefore, using the oncolytic virus to which human body has no or less immunity is critical to avoid rapid resistance to virus drugs in cancer therapy.

In order to overcome the defects of the virus as drug therapy, we have developed multiple RNA viruses as a group or a pool of drugs for cancer therapy. Flaviviruses compose of multiple serotype members and these members have different vaccination and/or epidemic history in certain areas worldwide. The members of flavivirus thus become good candidates as drugs. Because there are multiple serotypes, this allows us to selectively and alternatively administrate one of the oncolytic flaviviruses in the region where there is no vaccination or epidemic history. Furthermore, we can combine two or more different serotype stains into a therapy course to enhance treatment without repeatedly using a single virus drug, thus to reduce immune resistance to single virus treatment and to increase the virus drug efficacy. The combined use of multiple attenuated virus strains can overcome the defects of oncolytic virus treatment. At the same time, multiple administrations can be used alternately with oncolytic virus vectors that express functionally different factors in immunotherapy.

### SUMMARY

One aspect of the present disclosure provides a combination of immuno-oncolytic virus drugs that carry foreign gene fragment(s) derived from human to enhance a systemic immune response to malignancy.

Optionally, the combination of immuno-oncolytic virus drugs is a group of oncolytic viruses with different serotypes and contains a various foreign gene(s) encoding special activator(s) of the immune system for enhancement of immune response to cancers.

Optionally, since multiple flavivirus strains are available and each may carry different foreign gene fragment, they become a panel/pool of candidate drugs, which can be selectively and alternatively used to patients worldwide, where vaccination or epidemic history is of difference.

Further Optionally, the multiple flavivirus strains can selectively compose to form one or more treatment groups to meet the therapy needs. Thus, a combination of the immuno-oncolytic flavivirus drugs and alternative use of them can avoid immune resistance caused by virus antigens during the therapy and thus increase anti-cancer efficiency.

Optionally, the oncolytic virus includes flavivirus genus and/or positive-sense RNA viruses that are attenuated or mutated to less pathogenesis.

Optionally, the flavivirus has been modified through gene engineering not only carries additional active ingredients, exogenous gene fragment, but also is linked covalently to a DNA plasmid with a promoter nucleic acid sequence that regulates viral gene expression. The promoter may be derived from different tissues and species.

Optionally, a variety of exogenous gene fragments are integrated into the oncolytic virus genome, and are amplified and expressed to functional proteins.

Further Optionally, the exogenous gene fragments encode T cell co-stimulator and/or active factors that specifically activate various types of T cell subsets, and have a function to induce an immune response to cancers.

Optionally, the combination of the immuno-oncolytic virus drugs described above is administered to solid tumor in a form of biological macromolecule DNA, thus the replicable DNA can be used to a broad spectrum of tumors, including but not limit to melanoma, lung cancer, cervical cancer, prostate cancer, breast cancer, kidney cancer, colon cancer, or squamous cell cancer.

### BRIEF DESCRIPTION OF THE DRAWINGS

The following drawings are merely examples for illustrative purposes according to various disclosed embodiments and are not intended to limit the scope of the present disclosure.

FIG. 1 illustrates a diagram of the structure of oncolytic flavivirus drug, “1” denotes inserted foreign gene fragment; and “2” denotes viral genome.

FIG. 2 demonstrates expected results of the immunofluorescent assay (IFA) of the oncolytic WNV replication and foreign gene expression.

FIG. 3 illustrates that oncolytic WNV causes cancer cell CPE.

FIG. 4 illustrates that oncolytic WNV inhibits mouse tumor growth.

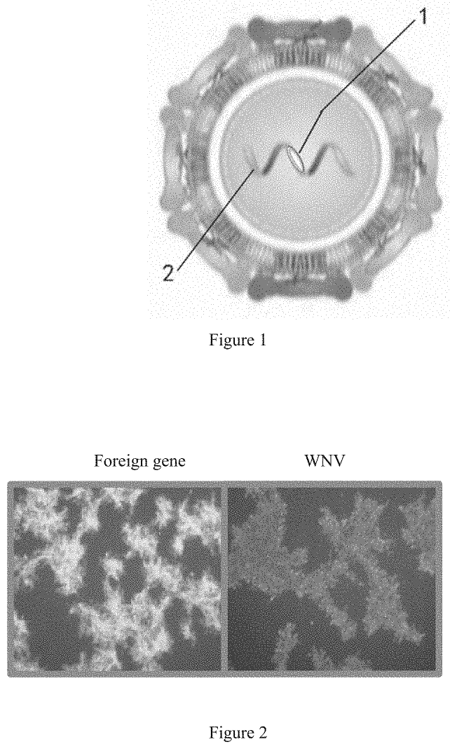

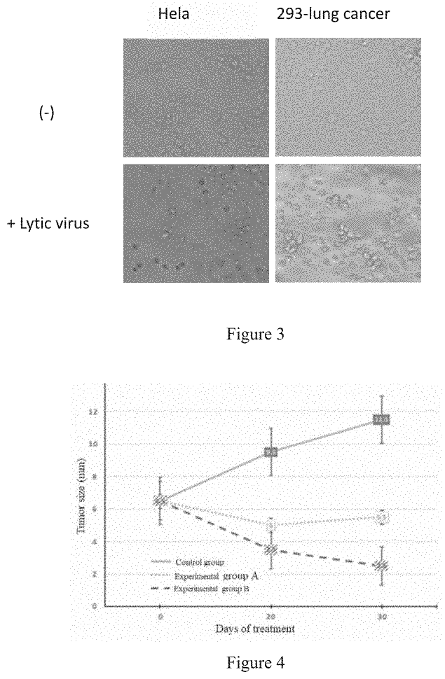

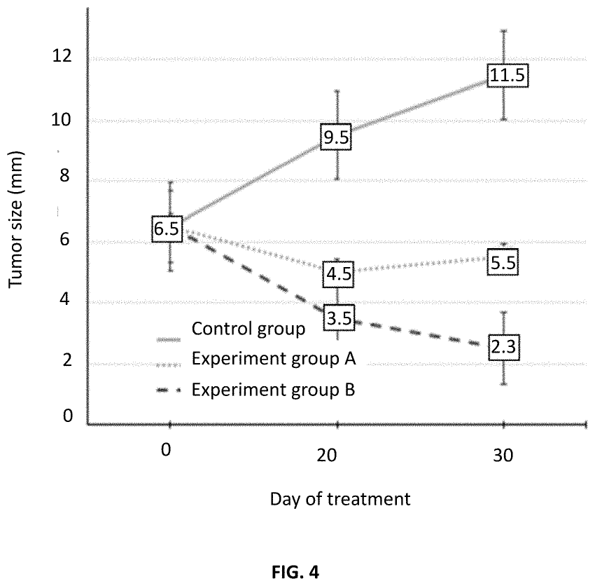

### DETAILED DESCRIPTION

The purpose of the present innovation is to overcome the defects of the existing oncolytic virus treatment and to provide novel pharmaceutics for immunotherapy of cancer. The application of a combination of immuno-oncolytic flavivirus drugs can reduce the immune resistance to oncolytic virus drugs and thus effectively enhance a systemic immune response to cancers.

The invention discloses a series of oncolytic flavivirus drugs that enhance a systemic immune response to treat cancer. The active ingredients of drugs comprise positive single stranded-RNA virus plus human gene fragments encoding unique T lymphocyte activators that specifically induce system immune response against cancers. The application of a combination of immuno-oncolytic flavivirus drugs can avoid inhibiting the oncolytic virus by the preformed immune defense and can reduce immune resistance for more round administrations, thus enhance the efficacy of cancer therapy.

The Flavivirus genus consists of more than 70 small, positive-sense, single-stranded RNA viruses transmitted by arthropods, in particular mosquitoes and ticks (Mathilde Laureti, et. al. 2018. Flavivirus Receptors: Diversity, Identity, and Cell Entry. Front Immunol. 9:2180). Forty species of the flavivirus family have been associated with human diseases. These include globally important human pathogens such as West Nile virus (WNV), Japanese encephalitis virus (JEV), dengue virus (DENV), Murray Valley encephalitis virus (MVE), tick-borne encephalitis virus (TBEV), Yellow Fever virus (YFV), and Zika virus (ZIKV).

The flaviviruses are small enveloped viruses (approximately 50 nm in diameter). Their genome of ˜11 kb contains a single open reading frame flanked by untranslated regions and encodes 3 structural proteins and 7 non-structural proteins. The ORF is flanked by a 5′ noncoding region (NCR) which is about 100 nucleotides in length and by a 3′-NCR which is 400 to 800 nucleotides in length. The mature virion features a surface densely covered with E glycoproteins and M proteins and a core consisting of capsid (C) protein and the RNA genome.

Flaviviruses are deposited into the skin epidermis by a mosquito bite where they encounter cells permissive to infection such as keratinocytes and skin dendritic cells (Langerhans cells). However, flaviviruses can replicate in a wide variety of species and have a broad cellular tropism, including nasal epithelial cells. Flaviviruses also infect many cell lines in vitro. The flavivirus envelope protein is the dominant antigen of eliciting neutralizing antibodies and plays an important role in inducing immunologic responses in the infected host. When flavivirus replicates in cells, similarly to other virus infections, the immune system will activate to respond to virus infection and simultaneously destroy virus-infected tumor cells, which is one of the mechanisms of cancer immunotherapy by oncolytic viruses. on the other hand, the rapidly induced immune response to virus infection becomes a hurdle to oncolytic therapy. The preformed immune responses can reduce treatment efficacy regardless of administrating quantity and times.

Live, attenuated vaccines have afforded the most effective and economical means of prevention and control of diseases, as illustrated by YF 17D and JE SA14-14-2 vaccines. Recent advances in recombinant DNA technology have made it possible to develop live attenuated flavivirus vaccines against flavivirus disease. Full-length cDNA clones allow the construction of infectious virus bearing attenuating mutations or deletions incorporated in the viral genome. It is also possible to create chimeric flaviviruses in which the structural protein genes for the target antigens of a flavivirus are replaced by the corresponding genes of another flavivirus. Encouraging results from preclinical and clinical studies have shown that several chimeric flavivirus vaccines have the safety profile and satisfactory immunogenicity and protective efficacy to warrant further evaluation in humans. The chimeric flavivirus strategy has led to the rapid development of novel live-attenuated vaccines against dengue, TBE, JE, and West Nile viruses. Currently approved available flavivirus vaccines include yellow fever (17D), Japanese encephalitis (SA14-14-2), Dengue viruses (Dengvaxia), and tick-borne encephalitis. These vaccine strains plus chimeric viruses can compose of a panel/bank of oncolytic flaviviruses.

The panel with multiple attenuated strains provides a choice to choose adequate serotype strain to administrate patients in a certain area and to use different stains as a combination in the treatment of cancer. Different flaviviruses can be selectively used (A) in the non-vaccination region and (B) in the non-epidemic regions where the population has no or lower immune pressure to oncolytic virus treatment. In some areas where flavivirus vaccination was performed or in an epidemic region, oncolytic viruses of different serotypes can be selectively used. For example, using oncolytic WNV (attenuated strain), instead of JEV, to treat cancer must be a practical and effective way to avoid high immunity to JEV where the broad population (e.g. in China) have gotten JEV vaccination. Whereas, application of oncolytic JEV vaccine strain in Northern American would be a good choice rather than using oncolytic yellow fever strain. And don't use oncolytic dengue viruses in Brazil where is the epidemic area of dengue infection and of high immunity to dengue virus. Therefore, selective and alternative application of multiple oncolytic flaviviruses has great advantages in order to avoid preformed immune pressure to virus antigens (that is the drug resistance) and to increase treatment efficiency through more than two administrations of different serotype of oncolytic viruses.

In addition, a combination of immuno-oncolytic virus drugs can be alternatively administrated in a course of cancer treatments which avoids immune resistance to a single oncolytic virus. Because of the pool of flaviviruses, we may design a good treatment plan: Using different serotypes of oncolytic flaviviruses in the cancer treatment cycle instead of continually using one serotype virus. Formulate several oncolytic viruses each carrying a different function of a foreign gene in a treatment course to maximize the activation of systemic immunity to tumors without causing drug resistance.

Altogether, the flavivirus genus as oncolytic virus ought to be great drug candidates owing to multiple stains available. Alternative application or combination of multiple attenuated strains can increase efficacy because of no cross-reaction among their serotypes thus avoids preformed immunity to virus antigen. Flaviviruses have similar gene structure and protein profile and smaller size of genome thus easy to manipulate for chimeric viruses with switched envelope protein and for integrating therapeutic genes. They do have broad tropism to infect mainly epithelial cells, which occur in 85% of cancers. Furthermore, RNA oncolytic virus drugs have more advantages than DNA oncolytic viruses: they have fewer viral proteins and the viral genes do not integrate into the host chromosome to cause oncogenic mutation and/or latent infection.

Moreover, the present disclosure provides flavivirus with new features that are the viruses contain exogenous gene fragment(s) encoding human T cell co-stimulator(s). Activating anergy T cells in the tumor microenvironment is critical to achieving a consistent response to cancer thus to prevent recurrent, metastasis, and to treat multiple tumors. Based on the “two signalings” theory, T cells activation requires not only major histocompatibility complex (MEW)—antigen but also needs T-cell co-stimulator(s). To meet the “two signalings” principle, the present disclosure takes the following strategies: Transplanting T cell co-stimulator, which originally expresses in APC, into tumor cells through flavivirus vector. This innovation speculates when the drug is injected into tumor tissue locally, the engineered live virus self-replicate in cancer cells and at the same time express T-cell activating factors that are not normally produced in cancer cells. When oncolytic viruses expose tumor antigens by repeatedly infecting and lysing tumor cells, simultaneously expressed T cells co-stimulator on the tumor membrane interacts with specific receptor/ligand on T cell and thus activating T-cells. Activation of specific subsets of T cells produces a series of chain reactions that mediate systemic immune responses, including inducing and activating killer T cells and memory T cells to re-identify and destroy tumor cells. Therefore, the key innovations of the present disclosure include: (1) using multiply attenuated RNA viruses as one type of oncolytic virus therapy; (2) transplanting T cell co-stimulatory molecules from APC to tumor cell through the vector; (3) on the surface of cancer cells, the expressed T cell co-stimulator provides a second signal to mediate a systemic immune response, thereby providing lasting immunity to cancer.

This strategy will not only improve the simple OV treatment that induces an indirectly immune response to cancer but also antagonize immune suppression produced by CTAL-4 or PD-1/PDL1 in cancer cells.

This innovation of expression T-cell co-stimulator in tumor cells thus is unique new immunotherapy. The oncolytic flavivirus drugs aim at the body's own immune system through T cell-mediated systemic immune response to kill cancer and may effectively eradicate recurrent cancer, metastasis, and multiple cancers which are failed at routine therapy by surgery, chimeric drugs, and radiation.

Since there are a number of T cell co-stimulators and lymphocyte factors playing different roles in the activation of the immune system, alternatively integrating each of these active factors into respect flavivirus vector can greatly expand anti-cancer arsenal. The combination of oncolytic flaviviruses thus include not only different serotypes of flavivirus but also comprise a group of lymphocyte factors carried by the flaviviruses. Therefore, the combination of different serotype of flavivirus with variant lymphocyte factors may obtain the best efficacy in cancer therapy.

Although some of the flaviviruses have been developed to be a vaccine for prevention virus infection, the new class of immuno-oncolytic flaviviruses claimed in this innovation has been genomic-modified to carry foreign gene fragment(s) for cancer therapy. The oncolytic virus contains all virus nucleotide sequences and codons for active lymphocyte factors. Therefore, the engineered oncolytic flaviviruses consist of new structural composition and new functional components, which may attenuate the virus and grant the oncolytic virus drugs a new function for immune therapy.

We have constructed the oncolytic flaviviruses vectors that carry a variety of T cell co-stimulator and lymphocyte factor genes, respectively. These oncolytic flaviviruses also have been tested in tumor mouse models and showed very good results. The 80% repression of tumor growth was observed in bilateral tumors of a mouse with only one side intratumor injection, reflecting a systemic immune response to the tumor that was not injected with the drug.

### EXAMPLE 1

Generation of Attenuated Flaviviruses

Considering the safety of using viruses as human drugs, it must be proven that these flaviviruses have been attenuated and will not cause human disease. In fact, many attenuated flaviviruses have been used as vaccines against human flavivirus infections. These attenuated live vaccines, including the yellow fever virus 17D strain and the Japanese encephalitis virus SA14-14-2 strain, have been vaccinated in a broad population in China and in the world, and have a very good safe history record. Another type of attenuated flaviviruses is an envelope hybrid flavivirus. In most cases, envelope hybrid flaviviruses consist of a flavivirus genome with heterologous envelope protein. Our research and many published data indicate that hybrid flaviviruses are attenuated automatically, comparing to their wild-type parental viruses and are also unlikely to return back to their parental virulence. For example, the recombinant-attenuated rWN/DEN4Δ30 virus is an envelope hybrid flavivirus of the wild-type West Nile virus (NY99) genome and attenuated live dengue (4rDEN4) Δ30 strains. The gene encoding the envelope protein of dengue-4 was replaced by the gene of the West Nile virus (WNV). In non-human primate and human trials, rWN/DEN4Δ30 has been demonstrated to be highly attenuated, and there is no evidence of neuroinvasive disease; and all monkeys vaccinated with a single dose of rWN/DEN4Δ30 showed moderate to high levels of WNV specificity NAB and completely protects against WNV NY99 infection. In addition, a comprehensive study of vaccine neuropathogenesis in the central nervous system of rhesus monkeys has shown that compared with the 17D reference vaccine for yellow fever, rWN/DEN4Δ30 has a higher degree of neurological attenuation.

Attenuation of WNV Virus:

Point mutations of amino acids of the envelope protein or mutations at the 3′ end of the non-coding region, both can result in neuro-attenuated flaviviruses. The inventors modified the infectious WNV cDNA and replaced five of neuron-related amino acids on WNV envelope. At the same time, the nucleotide sequence of the 3′ terminal stem-loop of the dengue type 2 replaced the wild-type WNV 3′ terminal stem-loop sequence and one or more mutations were also generated in the nucleotides of the WNV 3′ terminal stem-loop secondary structure. The transformed WNV showed attenuating characteristics. Subcutaneous injection of sensitive 3-week-old mice with WNV (MutE) did not cause mouse death and neurological diseases. When WNV envelope protein mutants (WN/Env5 and WNmutE-Env5) were injected into the brains of baby mice and showed a 1000-fold reduction in neurotoxicity.

Construction of an Attenuated Membrane Hybrid Flavivirus (ZIKA/WNV):

The inventor connected the cDAN of the entire WNV genome to a pBR322 plasmid vector containing a CMV promoter. Zika virus envelope gene fragment was synthesized by PCR with restriction enzyme sites at both ends. After digestion with the restriction enzyme, this fragment is ligated to the same site of the WNV cDNA. This recombinant plasmid was transformed into E. coli cells for amplification and purified by the routine process. The purified recombinant plasmid was transfected into animal or mosquito cells cultured in vitro, and the recombinant plasmid containing the CMV promoter transcribed infectious viral RNA in the cells. These viral RNAs replicate and produce a hybrid flavivirus (ZIKA/WNV) with a Zika virus envelope and WNV core protein. Animal experimental data show that the hybrid flavivirus with Zika virus envelope has lost the original WNV neurovirulence. The attenuated viruses thus can be safely used as oncolytic virus drugs as non-pathogenesis vector. In addition, the use of chimeric flaviviruses may also expand the diversity of oncolytic virus drugs, avoiding the resistance to a single serotype virus.

### EXAMPLE 2

Construction of infectious flaviviruses carrying foreign gene fragments: The disclosure relates to an oncolytic virus that contains artificially inserted exogenous gene fragments. These exogenous gene fragments are mainly human T cell co-stimulators. These non-viral exogenous gene fragments are inserted into different types of flavivirus genomes using conventional and commonly adopted genetic engineering methods. For an example of (YF) 17D/GFP, a flavivirus was integrated with a foreign gene without affecting viral replication. The 17D/GFP expresses the protein normally without affecting the virus replication. Immunoprecipitation and confocal laser scanning microscopy show the expression of GFP, which remains in the endoplasmic reticulum and is not secreted from infected cells. The virus was genetically stable during the 10th consecutive passage in Vero cells. As demonstrated by ELISA tests, the recombinant virus was able to elicit a neutralizing antibody response against YF and an antibody against GFP.

WNV Virus-Carrying T Cell Costimulatory Molecules

The attenuated WNV and hybrid ZIKA/WNV flaviviruses described in Example 1 were further genetically modified to contain a gene segment of the T cell co-stimulator. The conventional methods for engineering WNV vector include: PCR synthesizes human or mouse T cell gene fragments or GFP gene fragments; ligate these fragments to the attenuated WNV and hybrid ZIKA/WNV cDNA through restriction enzyme sites; colony selection of recombinants through restriction enzyme mapping and sequencing. The cloned recombinant plasmids were further examined for virus infection and foreign gene expression in cultured cells in vitro. As shown in FIG. 2, the immunofluorescence assay detected the WNV replication and the expression of the T cell co-stimulator. When lung or cervical cancer tumor cells were infected with these recombinant flaviviruses, the cells were dying from oncolytic virus infection after three days. Other characteristics of these recombinant flaviviruses include: they grow slower than wild-type strain with a peak of 1×10⁷ at 7 days; they have smaller plaques. The virus was genetically stable without losing the foreign gene expression after 10th consecutive passages in Vero cells. These results indicate that the insertion of foreign genes did not affect recombinant flaviviruses replication.

Through sensitive mouse experiments, there was no evidence of neuroinvasive disease and no mouse death. These data prove that the recombinant flaviviruses do not increase the variability but reduce toxicity (pathogenicity) as expected. Thus, they are safe for clinical application.

### EXAMPLE 3

Evaluation of the Inhibition Effects of Mouse Tumor by Oncolytic Virus:

A mouse stomach tumor (MFC) model was established. Mouse MFC tumor cells (supplied by Shanghai Fuxiang Biotechnology Co., Ltd.) were cultured in RPMI1640 medium containing 5% fetal bovine serum. MFC cells (5 million) growing at the logarithmic stage of growth were injected subcutaneously at two sides of the dorsal of 6-8 week old mice (C57BL/6). When MFC tumors on dorsal grow to an average diameter of 6.2 mm after about 8 days, the DNA form of recombinant WNV was injected to the right side tumor only.

Animal Groups:

1. Control group: 100 μl of PBS.

2. Experimental group A: 100 ug/100 ul WE/Hc86-(WNV virus carries human B7 gene fragment).

3. Experimental group B: 100 ug/100 ul WE/Mc86-(WNV virus carries mouse B7 gene fragment).

The experimental results are as follows:

1. In all 9 test mice, no symptoms were observed within 10 days after injection, and no death occurred within 30 days.

2. 20 days after the inoculation, the diameter of the tumor was measured: the average tumor diameter of the control group was 9.5 mm, the experimental group A was 5.0 mm, and the experimental group B was 3.5 mm.

3. 30 days after inoculation, diameter measurement: the average tumor diameter in the control group was 11.5 mm, the experimental group A was 5.5 mm, and the experimental group B was 2.5 mm.

4. Thirty days after the inoculation, tumor tissue sections and histochemical examination showed that there was a large number of T cell infiltration around the residual tumor in the experimental group B, which was several times more than the experimental group A.

Conclusion:

1. The inhibition of tumor growth was significantly different between groups. Compared with the control group, the tumor growth of the experimental group A was significantly suppressed, while the tumor of the experimental group B almost shrieked.

2. The difference in the number of T cell infiltration may be attributed to the activation response of the experimental group B mouse-derived B7 to the immune system.

3. Immune oncolytic virus (WE/Mc86) has a better cancer treatment effect than a non-specific oncolytic virus (WE/Hc86).

4. WNV oncolytic virus-carrying foreign gene fragments is safe as a therapeutic drug.

What is claimed is:

1. A combination of immuno-oncolytic virus drugs that carry immune gene fragment(s) derived from human to enhance a systemic immune response to malignancy, wherein therapeutic drugs comprise positive-sense single-stranded RNA (ssRNA) viruses with a plurality of different serotypes.

2. The combination of immuno-oncolytic virus drugs of claim 1, wherein the ssRNA viruses are the members of flavivirus genus, which contains more than ten different antigenicities of viruses to become a pool of anti-cancer drugs with a similar structure of genome as well as the similar constitution of protein molecules.

3. The combination of immuno-oncolytic virus drugs of claim 2, wherein the flavivirus genus comprises West Nile virus, Zika virus, 1-4 type of dengue viruses, yellow fever virus, Japanese encephalitis virus, and St. Louis encephalitis virus.

4. The combination of immuno-oncolytic virus drugs of claim 3, further comprising attenuated or non-attenuated strains, vaccine or non-vaccine strains with mutation either amino acids or non-coding regions, and/or chimeric virus strains, of which they are attenuated as like vaccines to the administration of human body without causing diseases.

5. The combination of immuno-oncolytic virus drugs of claim 1, wherein the different serotypes of viruses carry either one type of foreign genes or each virus may carry different foreign gene thus to expand the diversity and selectivity in the drug pool to increase the efficacy of cancer therapy.

6. The combination of immuno-oncolytic virus drugs of claim 5, wherein the foreign gene fragment(s) is covalently integrated into the genome of the viruses and is amplified and transcribed to express the active protein(s) as the virus replicates.

7. The combination of immuno-oncolytic virus drugs of claim 6, wherein the protein encoded by the foreign gene fragment(s) can be 50-100% molecular weight of a functional protein.

8. The combination of immuno-oncolytic virus drugs of claim 1, wherein the foreign gene fragment(s) encodes but not limit to human T cell co-stimulator and/or an activation factor that specifically activates different types of T cell subsets as active ingredients.

9. The combination of immuno-oncolytic virus drugs of claim 8, wherein human T cell co-stimulator(s) and activation factor(s)comprise CD80/86, ICOSL, OX40L, CD40, 4-1BBL, CD70, and CD30L, which are physiologically expressed in B cells and interacted to relative ligand/receptor in T cells.

10. The combination of immuno-oncolytic virus drugs of claim 1, wherein the oncolytic virus is connected covalently to a plasmid in a cDNA form and contains nucleic acid sequences of a promoter that regulates viral gene expression.

11. The combination of immuno-oncolytic virus drugs of claim 10, wherein the DNA drugs are manufactured through fermenting E. coli amplification, wherein when administrating the DNA drugs through intratumor injection, the DNA expresses and produces virus and T cell co-stimulator(s) to execute the therapy effect against cancer.

12. The combination of immuno-oncolytic virus drugs of claim 11, wherein the expressed T cell-activating factors are translocated into tumors and interact with T and B cells to stimulate a systemic immune response against cancer in vivo.

13. The combination of immuno-oncolytic virus drugs of claim 1 is used for immunotherapy of cancer, where the drugs may be administered either in one treatment or in more than two courses with alternative serotypes of flavivirus to avoid immune resistance to the oncolytic treatment resulted by the preformed immune response to the oncolytic virus.
